(19) United States
(12) Patent Application Publication
(10) Pub. No.: US 2010/0233227 A1
(43) Pub. Date: Sep. 16, 2010

(54) MEDICAL DEVICES HAVING CARBON DRUG RELEASING LAYERS

(75) Inventor: Jan Weber, Maastricht (NL)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/720,223

(22) Filed: Mar. 9, 2010

Related U.S. Application Data
(60) Provisional application No. 61/158,965, filed on Mar. 10, 2009.

(51) Int. Cl. A61F 2/82; A61F 2/00; A61P 9/00

(52) U.S. Cl. 424/422; 623/1.42

(57) ABSTRACT

According to various aspects of the invention, medical devices are provided, which comprise (a) a substrate, (b) a drug-containing layer disposed over the substrate, which contains one or more drugs and, optionally, one or more additional materials, and (c) a carbon layer disposed over the drug-containing layer.

### RELATED APPLICATIONS

This application claims priority from U.S. provisional application 61/158,965, filed Mar. 10, 2009 which is incorporated by reference herein in its entirety.

### FIELD OF THE INVENTION

The present invention relates generally to medical devices, and more particularly to implantable or insertable medical devices which contain carbon layers for drug release.

### BACKGROUND OF THE INVENTION

The delivery of a drug onto or within the body of a patient is common in the practice of modern medicine. In vivo delivery of drugs is often implemented using medical devices that may be temporarily or permanently placed at a target site within the body. These medical devices can be maintained, as required, at their target sites for short or prolonged periods of time, delivering drugs at the target site.

A one specific example, coronary stents such as those commercially available from Boston Scientific Corp. (TAXUS and PROMUS), Johnson & Johnson (CYPHER), and others are frequently prescribed use for maintaining blood vessel patency. These products are based on metallic expandable stents with biostable polymer coatings, which release antiproliferative drugs at a controlled rate and total dose for preventing restenosis of the blood vessel. One such device is schematically illustrated, for example, in FIGS. 1A and 1B. FIG. 1A is a schematic perspective view of a stent 100, having a structural design like that of FIG. 3 in U.S. Patent Pub. No. 2004/0181276. The stent 100 contains a number of interconnected struts 101. FIG. 1B is a cross-section taken along line b-b of strut 100s of stent 100 of FIG. 1A, and shows a stainless steel strut substrate 110 and a therapeutic-agent-containing coating 120, which encapsulates the stent strut substrate 110.

Various researchers have previously investigated the effects of ion beam treatment on surfaces. For example, A. Kondyurin, Institute of Polymer Research, Dresden, Germany, has proposed the structure shown in FIG. 2 for polyethylene upon ion beam treatment (N+, 20 keV). With reference to FIG. 2, an ion-beam-treated polyethylene film 200 is shown with the following four material regions (as one travels from the surface into the material): an oxidized interfacial region 240, a carbon layer 230 (which includes sp² and sp³ bonded carbon atoms, as discussed in more detail below), a crosslinked layer 220, and an unchanged layer 210.

### SUMMARY OF THE INVENTION

According to an aspect of the invention, medical devices are provided, which comprise (a) a substrate, (b) a drug-containing layer disposed over the substrate, which contains one or more drugs and, optionally, one or more additional materials, and (c) a carbon layer disposed over the drug-containing layer.

In certain embodiments, an additional rapidly dissolvable organic layer is disposed over the carbon layer. The additional rapidly dissolvable organic layer may be employed, for example, to protect the carbon layer from rupture, which could result in premature drug release. The additional rapidly dissolvable organic layer may be employed, for example, to hold in place rupturing elements, which act to rupture the carbon layer upon application of a compressive force to the drug-containing layer and the carbon layer, thereby increasing drug release.

In certain other embodiments, the drug-containing layer further contains one or more types of reinforcing elements, which act to resist compression of the drug-containing layer.

In still other embodiments, the drug-containing layer further contains one or more types of rupturing elements, which act to rupture the carbon layer upon application of a compressive force to the drug-containing layer and the carbon layer, thereby increasing drug release.

These and many other aspects, embodiments and advantages of the present invention will become readily apparent to those of ordinary skill in the art upon review of the Detailed Description and Claims to follow.

### BRIEF DESCRIPTION OF THE DRAWINGS

FIG. 1A is a schematic perspective view of a stent, in accordance with the prior art.

FIG. 1B is a schematic cross-sectional view of the stent of FIG. 1A, taken along line a-a in FIG. 1A.

FIG. 2 is a schematic illustration of various layers that may be present in a sample of polyethylene after ion beam treatment, in accordance with the prior art.

FIGS. 3-5 are schematic cross-sectional views of medical devices (or portions thereof), in accordance with various embodiments of the invention.

FIG. 6A is a schematic cross-sectional view of a medical device (or portion thereof), in accordance with an embodiment of the invention.

FIG. 6B is a schematic illustration of the device of FIG. 6A upon application of a compressive force to the device, in accordance with an embodiment of the invention.

FIG. 6C is a schematic illustration of the device of FIG. 6A upon expansion of the particles within the device, in accordance with an embodiment of the invention.

FIGS. 7-9 are schematic cross-sectional views of medical devices (or portions thereof), in accordance with various embodiments of the invention.

FIG. 10A is a schematic perspective view of a stent, in accordance with an embodiment of the invention.

FIG. 10B is a schematic cross-sectional view of the stent of FIG. 10A, taken along line a-a in FIG. 10A.

FIG. 11 is a schematic cross-sectional view of a medical device (or portion thereof), in accordance with an embodiment of the invention.

### DETAILED DESCRIPTION OF THE INVENTION

According to various aspects of the invention, implantable and insertable medical devices are provided that comprise (a) a substrate, (b) a drug-containing layer disposed over the substrate, which contains drug and, optionally, one or more additional materials, and (c) a carbon layer disposed over the drug-containing layer.

As used herein a “layer” of a given material has a thickness that is small (e.g., 10% or less, often much less) compared to both its length and width. As used herein a layer need not be planar, for example, taking on the contours of an underlying substrate. Layers can be discontinuous (e.g., patterned). Terms such as “film,” “layer” and “coating” may be used interchangeably herein.

The use of a carbon layer may be advantageous, for example, in that it can act as a barrier to drug release, for example, providing regulated drug release in some embodiments or providing complete or near complete prevention of drug release (unless and until the layer is ruptured, degraded, etc.) in other embodiments. In addition to acting as a drug release barrier, a carbon layer may also be advantageous, for example, in that it can promote cell/tissue growth.

In certain embodiments, an additional rapidly dissolvable layer is disposed over the carbon layer. The rapidly dissolvable layer may be employed, for example, to protect the carbon layer from rupture (e.g., in the case of a soft, organic rapidly dissolvable layer), which can result in premature drug release from the device. The rapidly dissolvable layer may also be employed, for example, to position one or more rupturing elements (e.g., a plurality of hard, sharp elements) proximate the carbon layer, which rupturing elements can promote (e.g., allow or increase) drug release from the device as discussed below.

In certain embodiments, the drug-containing layer further contains one or more types of rupturing elements. Such elements may be selected, for example, to rupture the carbon layer upon subjecting the device compressive force to the device (e.g. during the course of implantation or insertion), thereby promoting drug release. Such elements may also be selected, for example, to expand and rupture the carbon layer upon positioning of the device in vivo, thereby promoting drug release.

In certain embodiments, the drug-containing layer further contains one or more types of reinforcing elements. Such elements may be selected, for example, to resist compression of the drug-containing layer and consequently prevent damage to the carbon layer that might otherwise occur upon compression of the drug-containing layer (e.g., during implantation or insertion of said device). In this way, the reinforcing elements may prevent premature drug release from the device in certain embodiments of the invention.

Medical devices in accordance with the invention vary widely. Examples include implantable or insertable medical devices which may be selected, for example, from stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts, etc.), vascular access ports, dialysis ports, catheters (e.g., urological catheters or vascular catheters such as balloon catheters and various central venous catheters), guide wires, balloons, filters (e.g., vena cava filters and mesh filters for distil protection devices), embolization devices including cerebral aneurysm filler coils (including Guglielmi detachable coils and metal coils), embolic agents, septal defect closure devices, drug depots that are adapted for placement in an artery for treatment of the portion of the artery distal to the device, myocardial plugs, pacemakers, leads including pacemaker leads, defibrillation leads and coils, neurostimulation leads such as spinal cord stimulation leads, deep brain stimulation leads, peripheral nerve stimulation leads, cochlear implant leads and retinal implant leads, ventricular assist devices including left ventricular assist hearts and pumps, total artificial hearts, shunts, valves including heart valves and vascular valves, anastomosis clips and rings, tissue bulking devices, suture anchors, tissue staples and ligating clips at surgical sites, cannulae, metal wire ligatures, tacks for ligament attachment and meniscal repair, joint prostheses, spinal discs and nuclei, orthopedic prosthesis such as bone grafts, bone plates, fins and fusion devices, orthopedic fixation devices such as interference screws in the ankle, knee, and hand areas, rods and pins for fracture fixation, screws and plates for craniomaxillofacial repair, dental implants, or other devices that are implanted or inserted into the body.

As previously noted, in accordance with various aspects, the invention provides medical devices that comprise (a) a substrate, (b) a drug-containing layer disposed over the substrate and (c) a carbon layer disposed over the drug-containing layer.

Substrate materials may be selected, for example, from (a) organic materials (i.e., materials containing organic species, typically 50 wt % or more, for example, from 50 wt % to 75 wt % to 90 wt % to 95 wt % to 97.5 wt % to 99 wt % or more) such as polymeric materials (i.e., materials containing polymers, typically 50 wt % or more polymers, for example, from 50 wt % to 75 wt % to 90 wt % to 95 wt % to 97.5 wt % to 99 wt % or more) and biologics, (b) inorganic materials (i.e., materials containing inorganic species, typically 50 wt % or more, for example, from 50 wt % to 75 wt % to 90 wt % to 95 wt % to 97.5 wt % to 99 wt % or more), such as metallic inorganic materials (i.e., materials containing metals, typically 50 wt % or more, for example, from 50 wt % to 75 wt % to 90 wt % to 95 wt % to 97.5 wt % to 99 wt % or more) and non-metallic inorganic materials (i.e., materials containing non-metallic inorganic materials, typically 50 wt % or more, for example, from 50 wt % to 75 wt % to 90 wt % to 95 wt % to 97.5 wt % to 99 wt % or more), and (c) hybrid materials (e.g., hybrid organic-inorganic materials, for instance, polymer/metallic hybrids, polymer/ceramic hybrids, etc.).

Substrate materials may be biostable or bioerodable. As defined herein, a “biostable” material is one which remains intact for more than one year, up to the life of the patient (if not removed). Conversely, as defined herein, a “bioerodable” material is one which does not remain intact for more than one year after implantation or insertion of the medical device into the body (e.g., due to any of a variety of mechanisms including chemical breakdown, dissolution, etc.). “Intact” is defined herein as maintaining mechanical features that are at least at 75% of the initial mechanical values (e.g., mechanical strength) at the time of implantation. (The initial mechanical value may be, for example, the radial force needed to compress a stent or the mechanical strength of a spinal disk implant or the tensile strength of a suture wire, among many other examples.) For example, a bioerodable material may not remain intact for a period of 12 months, 6 months, 3 months, 1 month, 1 week or even 1 day, in some cases. Two examples of this spectrum are a vascular closing device whereby the functional time window needs to be less than a day after implantation and a stent intended for the SFA (superficial femoral artery) regions, which needs to mechanically support the vessel for at least 6 months.

In certain embodiments, a bioerodable material is selected that substantially completely disintegrates (e.g., 95 wt % or more of the material is removed) within a period of 12 months, 6 months, 3 months, 1 month, 1 week, or even 1 day in some cases.

Drug-containing layers include substantially pure drug layers (e.g., layers consisting essentially of one or more drugs) and layers that comprise drugs in combination with one or more additional materials. Examples of additional materials, among others, include blending materials (e.g., drug binders, drug diluents, etc.), reinforcing elements and rupturing elements, as discussed in more detail below.

Wherein the additional material is a blending material, examples include biostable and bioerodable materials, including organic materials, inorganic materials, and hybrid organic-inorganic materials. In many embodiments, blending material is an organic material.

Drug-containing layers may comprise, for example, from 5 wt % or less to 10 wt % to 25 wt % to 50 wt % to 75 wt % to 90 wt % to 95 wt % to 97.5 wt % to 99 wt % or more of one or more drugs.

“Drugs,” “therapeutic agents,” “pharmaceuticals,” “pharmaceutically active agents”, and other related terms may be used interchangeably herein. Various examples of drugs are set forth below.

Drug-containing layer thicknesses may vary widely, preferably ranging between 0.5 and 20 micrometers, for example, ranging from 0.5 to 1 to 2 to 5 to 10 to 20 micrometers. In various embodiments, drug-containing layer thickness is dictated by the desired drug dosage and the concentration of the drug within the drug-containing layer.

As previously indicated, one advantage of providing carbon layers over drug-containing layers, in accordance with the invention, is that carbon layers are amenable to regulating release of drug from the underlying drug-containing layers.

Another advantage of carbon layers is that they have surfaces that promote cell growth. Commonly, cells prefer attachment to hard surfaces such as those associated with carbon layers.

Depending on the nature and location of the device, such cells may be, for instance, epithelial cells, endothelial cells, muscle cells, connective tissue cells, and/or nerve cells, examples of which include among others: (a) squamous epithelial cells, such as non-keratinized squamous endothelial cells, for example, those lining the upper GI tract (e.g., cheek and esophagus) and lung alveoli, as well as the mesothelium lining of various major body cavities (e.g., peritoneal, pleural, pericardial) and the endothelium lining the heart, blood vessels, sinusoids and lymphatics, (b) cubodial epithelial cells, which frequently line glandular ducts, (c) columnar epithelial cells, such as those lining portions of the digestive tract (e.g., the stomach and small intestines), the female reproductive tract (e.g., the uterus and fallopian tubes), as well as numerous other body surfaces, (d) pseudostratified columnar epithelial cells, such as those lining portions of the respiratory tract (e.g., trachea) and ducts of the male reproductive system, (e) transitional epithelial cells, such as those lining the distensible walls of the urinary tract (e.g., the renal pelvis, ureters, bladder and urethra), (f) glandular epithelium, (g) smooth muscle cells, which lie beneath epithelial cells and endothelial cells in many body lumens such as many of those found in the vasculature, the genitourinary system, respiratory tract, and gastrointestinal tract, (h) cardiomyocytes, or (i) connective tissue cells such as fibroblasts.

As a specific example, upon implantation of a vascular stent, it is desirable in many instances that the stent become covered with vascular endothelial cells. A functional endothelial cell layer is known to be effective for purposes of reducing or eliminating inflammation and thrombosis, both of which can occur in conjunction with the implantation of a foreign body in the vasculature. See, e.g., J. M. Caves et al., J. Vasc. Surg. (2006) 44: 1363-8.

As defined herein a “carbon layer” is a layer that contains at least 75 mol % carbon atoms, for example, from 75 to 90 to 95 to 97 to 99 mol % carbon atoms or more.

In certain embodiments, the carbon layer is a diamond-like carbon (DLC) layer. Diamond-like carbon is generally hard, amorphous, and chemically inert. Diamond-like carbon is known to be biocompatible and is relatively non-conductive.

As used herein, a “diamond-like carbon layer” is one that contains a mixture of sp² bonded carbon atoms (as in graphite) and sp³ bonded carbon atoms (as in diamond). Typically, the sp³ fraction (i.e., the number of sp³ carbons÷[the number of sp³ carbons+the number of sp² carbons]) of the diamond-like carbon layer is at least 10%. Thus, diamond-like carbon layers for use in the present invention may comprise an sp³ fraction ranging from 10% or less to 20% to 30% to 40% to 50% to 60% to 70% to 80% to 90% or more, preferably 50% or more. In this regard, the term “tetrahedral amorphous carbon” (ta-C) is sometimes used to refer to diamond-like carbon with a high degree of sp³ bonding (e.g., 80% or more).

Properties of diamond-like carbon typically vary with the sp³ fraction. For example an sp³ fraction ranging from 10% to 80% has been reported to correspond to a change in surface Young's modulus from about 10 GPa to about 90 GPa.

Diamond-like carbon may contain, for example, up to 25 mol % (e.g., from 25 to 10 to 5 to 2 to 1 mol % or less) of other elements besides carbon (e.g., impurities, etc.), including H, O, N and P, among others.

Diamond-like carbon layers range widely in thickness, for example, ranging from 5 nm or less up to several μm in thickness, more typically ranging from 10 nm or less to 25 nm to 50 nm to 100 nm to 250 nm to 500 nm or more in thickness.

As previously noted, in various embodiments, the drug-containing layers are provided with reinforcing elements and/or rupturing elements. Such elements may be biostable or bioerodable.

In certain embodiments, the drug-containing layers are provided with hard reinforcing elements and/or rupturing elements. As defined herein, a “hard” element is one that is formed from a material having a surface Young's modulus of at least 5 times that of the surrounding matrix (e.g., ranging from 5 to 10 to 25 to 50 to 100 times or more). Such materials may be selected, for example, from suitable organic and inorganic materials described below. A few specific examples include graphite, silicon, silicon dioxide, silicon nitride, carbon nitride, metals, ceramics including metal oxides, metal nitrides, metal carbides, metal carbonates, and metal boride, salt crystals, sugar crystals and biological materials such as bone, among many others.

In various embodiments the hard reinforcing elements and hard rupturing elements are in the form of particles. Particle size may vary substantially, so long as the particles provide the desired functionality. In various embodiments, particle size may range from 5% to 10% to 25% to 50% to 75% to 90% up to 100% of the drug layer thickness.

Particle aspect ratios may also vary substantially. For example, particles may have aspect ratios ranging from 1 to 2 to 5 to 10 to 100 or more (e.g., for fibers). “Aspect ratio” is defined herein as the greatest dimension of the particle divided by the smallest dimension of the particle, where the particle dimension is selected from particle length, height and width. (For a sphere, length width and height equal to the diameter, for a cylindrical fiber, the width and height equal the diameter.)

In some embodiments, one or more hard reinforcing elements are provided within the drug-containing layer, in order to resist compression of the drug-containing layer and thereby prevent damage to the overlying carbon layer, for example, due to application of a compressive force to the drug-containing layer and the carbon layer (e.g., compressive forces of the magnitude that are experienced during medical device production or during implantation or insertion of the device, for instance, during the introduction of the medical device into another medical device to assist placement in the body, etc.).

In certain embodiments, the one or more hard reinforcing element is in the form of one or more connected/monolithic structures. Examples of such structures include fiber networks, screens, bucky paper, and porous membranes (e.g., having pore sizes ranging from 50 to 1000 nm), among various other possibilities.

In certain other embodiments, multiple, hard reinforcing elements are provided in the form of reinforcing particles. Such particles preferably have smooth surfaces, for example, having spherical and/or oval cross-section. Such smooth particles include elongated particles of spherical and/or oval cross-section (including fibers). Such smooth particles include spheroidal particles, including spherical (to the eye) particles and other spheroids such as prolate spheroids (elongated spheres), and oblate spheroids (flattened spheres). In certain embodiments, the particles are “monodisperse” spheres, where are defined herein as a group of spherical particles that are of substantially the same size (i.e., having a size distribution such that at least 95% up to 100% of the spheres have diameters that are within 10% of one another).

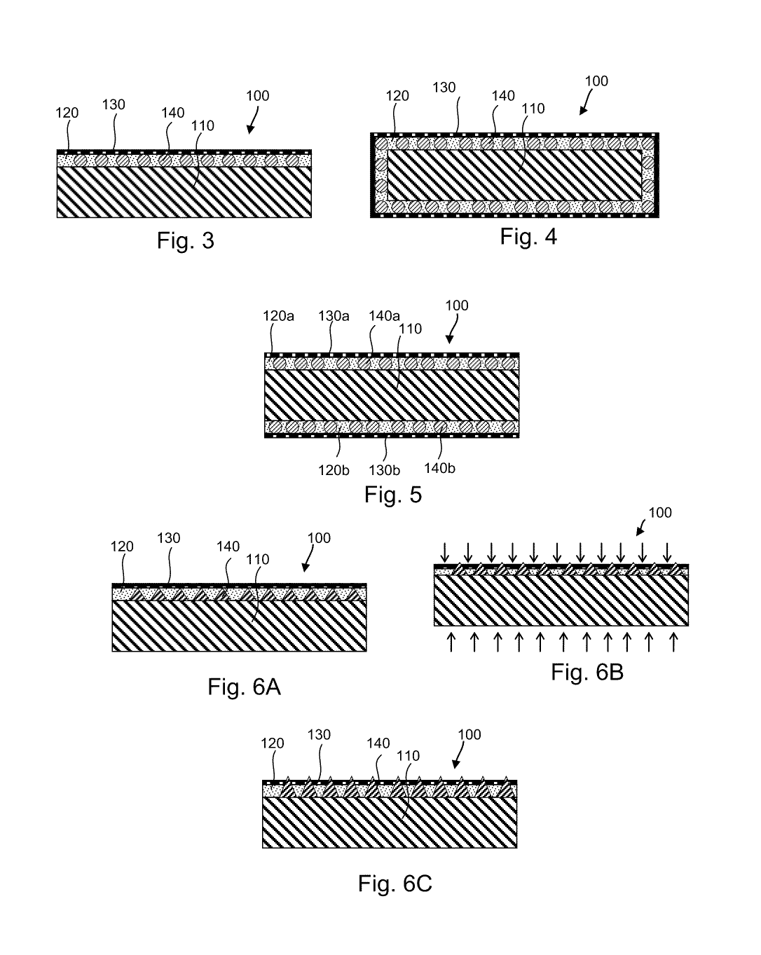

Turning now to the drawings, in FIG. 3 there is shown a schematic cross-sectional view of a medical device 100 (or a portion of a medical device) in accordance with the present invention. The device 100 includes a substrate 110, a drug-containing layer 120 disposed over the substrate 110, and a carbon layer 130 disposed over the drug-containing layer 120. Dispersed within the drug containing layer 120 are reinforcing particles 140, which support the carbon layer 130 and resist compression of the drug containing layer 120.

In one particular embodiment, the medical device 100 of FIG. 3 is a stent, with the view shown being a schematic representation of a cross-section of a stent strut 110. The stent strut 110 may be formed for example from a material such as tantalum or stainless steel 316L, among other materials. The reinforcing particles 140 may be, for example, monodisperse spheres of silica, ranging from 200 to 8000 nm microns in diameter as can be obtained from MicroParticles GmbH, Volmerstr. 9A, UTZ, Geb.3.5.1, D-12489 Berlin. The carbon layer 130 may range, for example, from 100 to 1000 nm in thickness. The drug containing layer 120 may be disposed, for example, over the outer abluminal surface of the stent strut and comprise an antirestenotic drug (e.g., paclitaxel, olimus family drug, etc.). Conversely, the drug containing layer 120 may be disposed over the inner luminal surface of the stent strut and may comprise, for example, a drug that promotes vascular endothelial cell growth (e.g., VEFG-1) and/or an antithrombotic drug (e.g., aspirin, warfarin or ticlopidine). By providing a carbon layer on the inner surface of the stent, endothelial cell growth is promoted on the inner surface. The drug-containing layer 120 may range, for example, from 0.5 to 20 microns in thickness, with the thickness depending, for example, upon the desired drug dose and whether or not an additional blending material, such as a binder or diluent, is provided in the layer (in which case the thickness of the layer may be increased). In a particularly preferred embodiment, the non-particle portion of the drug containing layer may comprise, for example, from 95 wt % to 100 wt % of the drug.

In other embodiments, such as that shown in schematic cross-section in FIG. 4, the medical device 100 (or portion of a medical device) includes a substrate 110 and a drug-containing layer 120 disposed over the substrate 110, which surrounds/encapsulates the substrate. As in FIG. 3, a carbon layer 130 is disposed over the drug-containing layer 120, and reinforcing particles 140 are dispersed within the drug containing layer 120, which support the carbon layer 130 and resist compression of the drug containing layer 120.

In one particular embodiment, the medical device 100 of FIG. 4 is a stent, with the view shown being a schematic representation of a cross-section of a stent strut 110. The stent strut 110, drug-containing layer 120, reinforcing particles 140 and carbon layer 130 may be dimensioned and formed from materials like those described above for FIG. 3, among many other possibilities. With regard to particular drug employed, the drug-containing layer 120 may contain one, two or all three of the following: an antirestenotic drug, an antithrombotic drug and a vascular endothelial cell growth promoting drug.

In still other embodiments, such as that shown in schematic cross-section in FIG. 5, the medical device 100 (or portion of a medical device) includes a substrate 110, first and second drug-containing layers 120a and 120b disposed over different portions of the substrate 110 surface, first and second carbon layers 130a and 130b disposed over the drug-containing layers 120a and 120b, respectively. First reinforcing particles 140a are dispersed within the first drug containing layer 120a and second reinforcing particles 140b are dispersed within the second drug containing layer 120b.

The first and second drug-containing layers 120a and 120b may be of the same or different composition and of the same or different thickness. Similarly, the first and second carbon layers 130a and 130b may be of the same or different composition (e.g., same or differing sp³ fraction) and of the same or different thickness. Also, the first and second reinforcing particles 140a and 140b may be of the same or different composition and of the same or different diameter.

In one particular embodiment, the medical device 100 of FIG. 5 is a stent, with the view shown being a schematic representation of a cross-section of a stent strut 110. The stent strut 110, drug-containing layers 120a, 120b, the particles 140a, 140b and carbon layers 130a, 130b may be dimensioned and formed from materials like those described above for the stent strut 110, drug-containing layer 120, particles 140 and carbon layer 130 of FIG. 3, among many other possibilities. The first drug containing layer 120a may be disposed, for example, over the outer abluminal surface of the stent strut 110 and comprise an antirestenotic drug. The second drug containing layer 120b may be disposed, for example, over the inner luminal surface of the stent strut 110 and may comprise a drug that promotes vascular endothelial cell growth and/or an antithrombotic drug.

In the preceding embodiments, the solid particles are selected such that they prevent the drug-containing layer from being compressed, in order to help prevent the carbon layer from being damaged.

In other embodiments, rupturing elements are selected such that they rupture the carbon layer, thereby promoting (e.g., allowing or increasing) drug release.

For example, in some embodiments, rupturing elements are provided within the drug-containing layer to rupture the overlying carbon layer upon application of a compressive force across the drug-containing layer and the carbon layer. In such embodiments, the rupturing elements may comprise hard, sharp particles. As defined herein, a “hard, sharp particle” is one having a point or edge whose dimensions and hardness enable the carbon layer to be ruptured when the particle is pressed against the carbon layer at a specified point during the device implantation process (e.g., for a stent, during balloon inflation). Also, the higher the concentration/density of the particles, the sharper the particles have to be to concentrate the force needed to rupture the carbon layer (analogous to the well-known “bed of nails” magic trick). Where provided beneath the carbon layer, the hard sharp particles should allow the carbon layer to be ruptured, without delaminating the carbon layer from the underlying structure during rupture. Examples of sharp particles include particles having sharp edges, particles having sharp corners, spiked particles, and so forth. Examples of sharp particles include regular particles such as pyramidal particles and irregular particles such as shards (e.g., crushed glassy materials), dendritic particles, and so forth.

In certain embodiments, a “sharp” particle will be a particle having a width that is substantially larger than 1 micrometer, with one or more points or edges with a radius of curvature of less than 1 micrometer. Preferably, the particles have multiple sides with sharp points or edges, such that the rupturing is independent of the orientation of the particle in the matrix.

As another example, in some embodiments, rupturing elements are selected to expand in vivo (e.g., upon contacting bodily fluid). The expansion generates a force that is sufficient to rupture (e.g., crack) the carbon layer. For example, the rupturing elements may be formed of bioerodable metal elements (e.g., bioerodable metal particles) that expand in vivo as a result of corrosion processes, resulting in rupture of the carbon layer. As another example, the rupturing elements may be formed of hydrogel elements (e.g., hydrogel particles) which expand as a result of hydration in vivo, resulting in breakage of the carbon layer.

For example, turning now to FIG. 6A, there is shown a schematic cross-sectional view of a medical device 100 (or a portion of a medical device) in accordance with the present invention. The device 100 includes a substrate 110, a drug-containing layer 120 disposed over the substrate 110, and a carbon layer 130 disposed over the drug-containing layer 120. The device further includes rupturing particles 140 dispersed within the drug containing layer 120.

As noted above, in some embodiments, the rupturing particles 140 are hard sharp particles, which concentrate force during compression and cause the carbon layer 130 to rupture upon application of external pressure. This rupturing effect is schematically shown in FIG. 6B, which shows the medical device 100 of FIG. 6A (in an embodiment where the particles 140 are hard, sharp particles) after application of a compressive force on the device.

In other embodiments, rupturing particles 140 are employed which expand in vivo to create pressure that causes the carbon layer 130 break. This rupturing effect is schematically shown in FIG. 6C, which shows the medical device 100 of FIG. 6A (in an embodiment where the particles 140 are expandable particles), after in vivo expansion of the particles 140. In these embodiments, either sharp or smooth particles may be employed to rupture the carbon layer 130, although sharp particles may be preferred to prevent or minimize delamination of the carbon layer 130 from the underlying a drug-containing layer 120.

In one particular embodiment, the medical device 100 of FIG. 6A is a stent, with the view shown being a schematic representation of a cross-section of a stent strut 110. The stent strut 110, drug-containing layer 120 and carbon layer 130 may be dimensioned and may be formed from materials like those described above for the stent strut of FIG. 3, among many other possibilities. The drug containing layer 120 may be disposed, for example, over the outer abluminal surface of the stent strut and comprise an antirestenotic drug. Conversely, the drug containing layer 120 may be disposed, for example, over the inner luminal surface of the stent strut and may comprise a drug that promotes vascular endothelial cell growth and/or an antithrombotic drug. (In other embodiments, a first drug containing layer may be disposed over the outer abluminal surface of the stent strut and comprise an antirestenotic drug and a second drug containing layer 120 may be disposed, over the inner luminal surface of the stent strut and comprise a drug that promotes vascular endothelial cell growth and/or an antithrombotic drug.)

Examples of particles 140 that may expand in vivo to rupture the carbon layer 130 include corrodible metals such as magnesium-iron alloys and hydrogels such as polyvinyl alcohol, among many others.

Examples of hard, sharp particles 140 which can rupture the carbon layer 130 upon compression include calcium carbonate crystals having an irregular shape. Where disposed on an outer abluminal surface of the stent, a compressive force may be applied by the vessel wall, upon expansion of the stent. Such an embodiment is desirable, for example, in that the layers are compressed to a greater degree, and thus drug is released to a greater degree, in areas of vascular obstruction. Where disposed on an inner luminal surface of the stent, a compressive force may be applied by the balloon upon expansion of the stent.

The devices of FIG. 6A and FIG. 6C are analogous to the device of FIG. 3, except that the reinforcing particles of FIG. 3 are replaced with rupturing particles in FIG. 6A and FIG. 6C. Additional devices analogous to those of FIGS. 4 and 5 may be also formed, in which the reinforcing particles of FIGS. 4 and 5 are replaced with rupturing particles such as those described in conjunction with FIG. 6A and FIG. 6C.

In other embodiments of the invention, an additional rapidly dissolvable layer is provided over the carbon layer. In some embodiments, such a layer may be provided, for example, to protect the carbon layer until the time that the device is implanted or inserted into a subject. In other embodiments, such a layer may be provided, for example, to temporarily hold in place hard, sharp rupturing elements, which are used to rupture the carbon layer upon implantation or insertion into a subject.

Because the layer rapidly dissolves (i.e., over a period of less than or equal to 24 hours, for example, ranging from 24 hours to 12 hours to 6 hours to 4 hours to 2 hours to 1 hour or less) such material does not, for example, interfere with cell growth in vivo. In fact, drugs that promote in vivo cell growth may be included in the layer in certain embodiments. By selecting materials that dissolve without any associated chemical reaction, one can avoid the formation of decomposition products that might otherwise interfere with the local environment (e.g., acidic products such as those associated with degradation of biodegradable polymers such as hydroxy acids, etc.).

Such rapidly dissolvable layers may range, for example, from 1 to 20 microns in thickness. Preferred materials for such layers include frozen materials such as ice or biocompatible organic materials, for instance, suitable members selected from the organic materials described below, among others. Particularly preferred materials include polysaccharides, heparin, albumin, fibrinogen, elastin, biocompatible waxes, esters, including fatty acid and fatty alcohol esters, such as isopropyl myristate, diisopropyl adipate, isopropyl laurate, isopropyl linoleate, isopropyl palmitate or cetyl palmitate, among others.

Turning now to FIG. 7, there is shown a schematic cross-sectional view of a medical device 100 (or a portion of a medical device) in accordance with the present invention. The device 100 includes a substrate 110, a drug-containing layer 120 disposed over the substrate 110, a carbon layer 130 disposed over the drug-containing layer 120, and a rapidly dissolvable layer 150 disposed over the carbon layer 130.

In one particular embodiment, the medical device 100 of FIG. 7 is a stent, with the view shown being a schematic representation of a cross-section of a stent strut 110. The stent strut 110 may be formed for example from a material such as tantalum or stainless steel. The carbon layer 130 may range, for example, from 50 nm to 1000 nm in thickness. The rapidly dissolvable layer 150 may be formed, for example, from a material such as isopropyl laurate, and may range, for example, from 1 micrometer to 5 micrometers in thickness. The drug containing layer 120 may be disposed, for example, over the outer abluminal surface of the stent strut and comprise an antirestenotic drug. Conversely, the drug containing layer 120 may be disposed, for example, over the inner luminal surface of the stent strut and may comprise a drug that promotes vascular endothelial cell growth and/or an antithrombotic drug. The drug-containing layer 120 may range, for example, from 500 nm to 20 microns in thickness, with the thickness depending upon the desired drug dose and whether or not an additional blending material is provided in the layer. In one preferred embodiment, the drug containing layer may comprise, for example, from 95% to 100% of the drug.

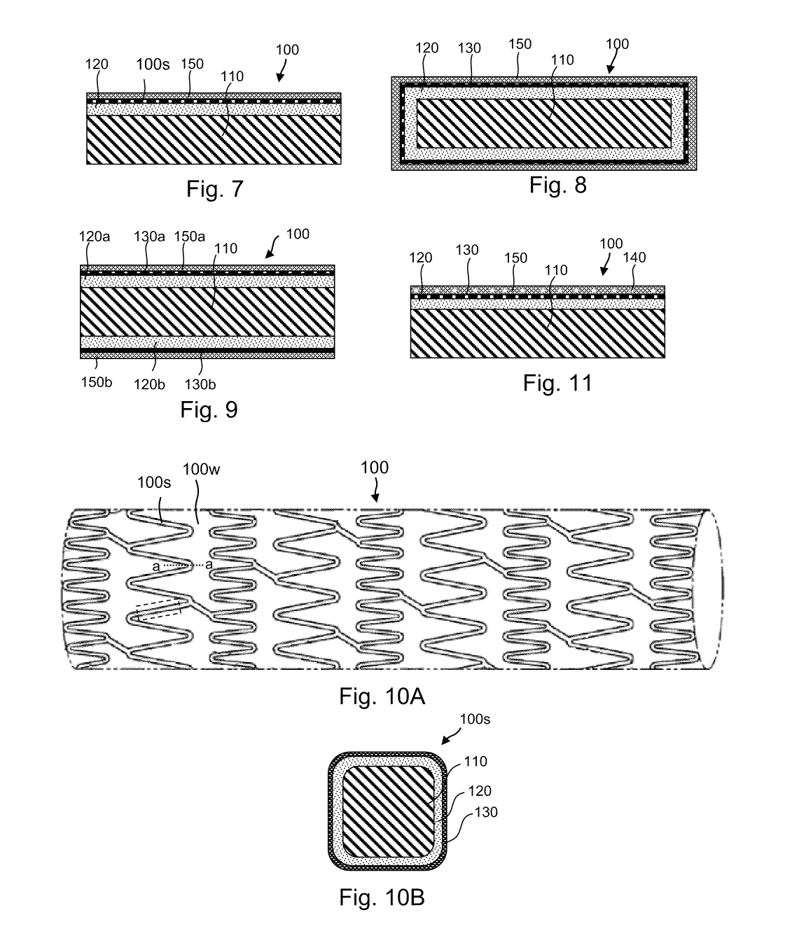

In other embodiments, such as that shown in schematic cross-section in FIG. 8, the medical device 100 (or portion of a medical device) includes a substrate 110, a drug-containing layer 120 disposed over the substrate 110 and surrounding/encapsulating the substrate. As in FIG. 7, a carbon layer 130 is disposed over the drug-containing layer 120, and a rapidly dissolvable layer 150 disposed over the carbon layer 130.

In a particular embodiment, the medical device 100 of FIG. 8 is a stent, with the view shown being a schematic representation of a cross-section of a stent strut 110. The drug-containing layer 120, carbon layer 130 and rapidly dissolvable layer 150 may be dimensioned and may be formed from materials like those described above for the stent strut of FIG. 7. With regard to particular drug employed, the drug-containing layer 120 may contain one, two or all three of the following, among others: an antirestenotic drug, an antithrombotic drug and a vascular endothelial cell growth promoting drug.

In other embodiments, such as that shown in schematic cross-section in FIG. 9, the medical device 100 (or portion of a medical device) includes a substrate 110, first and second drug-containing layers 120a and 120b disposed over different portions of the substrate 110, first and second carbon layers 130a and 130b disposed over the drug-containing layers 120a and 120b, respectively, and first and second rapidly dissolvable layer 150a and 150b disposed over the carbon layers 130a and 130b, respectively. The first and second drug-containing layers 120a and 120b may be of the same or different composition and of the same or different thickness. Similarly, the first and second carbon layers 130a and 130b may be of the same or different composition and of the same or different thickness. Also, the first and second rapidly dissolvable layers 150a and 150b may be of the same or different composition and of the same or different thickness.

In one particular embodiment, the medical device 100 of FIG. 9 is a stent, with the view shown being a schematic representation of a cross-section of a stent strut 110. The drug-containing layers 120a, 120b, carbon layers 130a, 130b and rapidly dissolvable layers 150a, 150b may be dimensioned and may be formed from materials like those described above for the drug-containing layer 120, carbon layer 130 and rapidly dissolvable layer 150 of the stent strut of FIG. 7. The first drug containing layer 120a may be disposed, for example, over the outer abluminal surface of the stent strut 110 and may comprise an antirestenotic drug (e.g., paclitaxel or an olimus family drug). The second drug containing layer 120b may be disposed, for example, over the inner luminal surface of the stent strut 110 and may comprise a drug that promotes vascular endothelial cell growth and/or an antithrombotic drug.

In certain embodiments, hard, sharp particles 140 are included in the rapidly dissolvable layer 150 as shown in FIG. 11. FIG. 11 is otherwise analogous to FIG. 7. Such particles 150 may be introduced, for example, to penetrate the carbon layer upon application of external pressure, thereby promoting (e.g., allowing or increasing) drug release. Examples of suitable hard, sharp particles 140 are discussed elsewhere herein. In the case where such particles are applied to the outer surface of a vascular stent, such particles may also penetrate the surrounding cell wall, increasing drug delivery to the same.

As previously indicated, a variety of materials can be used in the invention as substrate materials, rapidly dissolvable materials, blending materials and reinforcing elements and rupturing elements. Such materials may be selected from suitable members of the metallic, inorganic non-metallic, organic and hybrid materials listed below.

Specific examples of metallic materials may be selected, for example, from biostable metals such as gold, iron, niobium, platinum, palladium, iridium, osmium, rhodium, titanium, tantalum, tungsten, ruthenium, zinc, and magnesium, among others, biostable alloys such as those comprising iron and chromium (e.g., stainless steels, including platinum-enriched radiopaque stainless steel), niobium alloys, titanium alloys, alloys comprising nickel and titanium (e.g., Nitinol), alloys comprising cobalt and chromium, including alloys that comprise cobalt and chromium (e.g., Elgiloy alloys), alloys comprising nickel, cobalt and chromium (e.g., MP 35N), alloys comprising cobalt, chromium, tungsten and nickel (e.g., L605), alloys comprising nickel and chromium (e.g., inconel alloys), bioerodable metals such as magnesium, zinc and iron, and bioerodable alloys including alloys of magnesium, zinc and/or iron (and their alloys with combinations of Ce, Ca, Al, Zr, La and Li), among others (e.g., alloys of magnesium including its alloys that comprises one or more of Fe, Ce, Al, Ca, Zn, Zr, La and Li, alloys of iron including its alloys that comprise one or more of Mg, Ce, Al, Ca, Zn, Zr, La and Li, alloys of zinc including its alloys that comprise one or more of Fe, Mg, Ce, Al, Ca, Zr, La and Li, etc.).

Specific examples of inorganic non-metallic materials may be selected, for example, from biostable and bioerodable materials containing one or more of the following: nitrides, carbides, borides, and oxides of various metals, including those above, among others, for example, aluminum oxides and transition metal oxides (e.g., oxides of iron, zinc, magnesium, titanium, zirconium, hafnium, tantalum, molybdenum, tungsten, rhenium, niobium, and iridium); silicon; silicon-based ceramics, such as those containing silicon nitrides, silicon carbides and silicon oxides (sometimes referred to as glass ceramics); various metal- and non-metal-phosphates, including calcium phosphate ceramics (e.g., hydroxyapatite); other bioceramics; calcium carbonate; carbon; and carbon-based, ceramic-like materials such as carbon nitrides.

Specific examples of organic materials include polymers (biostable or bioerodable) and other high molecular weight organic materials, and may be selected, for example, from suitable materials containing one or more of the following, among others: polycarboxylic acid homopolymers and copolymers including polyacrylic acid, alkyl acrylate and alkyl methacrylate homopolymers and copolymers, including poly(methyl methacrylate-b-n-butyl acrylate-b-methyl methacrylate) and poly(styrene-b-n-butyl acrylate-b-styrene) triblock copolymers, polyamides including nylon 6,6, nylon 12, and polyether-block-polyamide copolymers (e.g., Pebax® resins), vinyl homopolymers and copolymers including polyvinyl alcohol, polyvinylpyrrolidone, polyvinyl halides such as polyvinyl chlorides and ethylene-vinyl acetate copolymers (EVA), vinyl aromatic homopolymers and copolymers such as polystyrene, styrene-maleic anhydride copolymers, vinyl aromatic-alkene copolymers including styrene-butadiene copolymers, styrene-ethylene-butylene copolymers (e.g., a poly(styrene-b-ethylene/butylene-b-styrene (SEBS) copolymer, available as Kraton® G series polymers), styrene-isoprene copolymers (e.g., poly(styrene-b-isoprene-b-styrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as poly(styrene-b-isobutylene-b-styrene) or SIBS, which is described, for instance, in U.S. Pat. No. 6,545,097 to Pinchuk et al.), ionomers, polyesters including polyethylene terephthalate and aliphatic polyesters such as homopolymers and copolymers of lactide (which includes d-,1- and meso-lactide), glycolide (glycolic acid) and epsilon-caprolactone, polycarbonates including trimethylene carbonate (and its alkyl derivatives), polyanhydrides, polyorthoesters, polyether homopolymers and copolymers including polyalkylene oxide polymers such as polyethylene oxide (PEO) and polyether ether ketones, polyolefin homopolymers and copolymers, including polyalkylenes such as polypropylene, polyethylene, polybutylenes (such as polybut-1-ene and polyisobutylene), polyolefin elastomers (e.g., santoprene) and ethylene propylene diene monomer (EPDM) rubbers, fluorinated homopolymers and copolymers, including polytetrafluoroethylene (PTFE), poly(tetrafluoroethylene-co-hexafluoropropene) (FEP), modified ethylene-tetrafluoroethylene copolymers (ETFE) and polyvinylidene fluoride (PVDF), silicone homopolymers and copolymers including polydimethylsiloxane, polyurethanes, biopolymers such as polypeptides, proteins, glycoproteins, polysaccharides, fibrin, fibrinogen, collagen, elastin, chitosan, gelatin, starch, and glycosaminoglycans such as hyaluronic acid; as well as blends and further copolymers of the above.

As noted above, a variety of drugs can be used in the invention.

Exemplary drugs for use in connection with the present invention include: (a) anti-thrombotic agents such as heparin, heparin derivatives, urokinase, clopidogrel, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (O) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, diclomine), (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.), (z) selective estrogen receptor modulators (SERMs) such as raloxifene, lasofoxifene, arzoxifene, miproxifene, ospemifene, PKS 3741, MF 101 and SR 16234, (aa) PPAR agonists, including PPAR-alpha, gamma and delta agonists, such as rosiglitazone, pioglitazone, netoglitazone, fenofibrate, bexaotene, metaglidasen, rivoglitazone and tesaglitazar, (bb) prostaglandin E agonists, including PGE2 agonists, such as alprostadil or ONO 8815Ly, (cc) thrombin receptor activating peptide (TRAP), (dd) vasopeptidase inhibitors including benazepril, fosinopril, lisinopril, quinapril, ramipril, imidapril, delapril, moexipril and spirapril, (ee) thymosin beta 4, (ff) phospholipids including phosphorylcholine, phosphatidylinositol and phosphatidylcholine, (gg) VLA-4 antagonists and VCAM-1 antagonists.

Several preferred drugs include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, zotarolimus, biolimus, Epo D, dexamethasone, estradiol, halofuginone, cilostazole, geldanamycin, alagebrium chloride (ALT-711), ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well derivatives of the forgoing, among others.

Numerous drugs, not necessarily exclusive of those listed above, have been identified as candidates for vascular treatment regimens, for example, as agents targeting restenosis (antirestenotics). Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists such as bosentan, sitaxsentan sodium, atrasentan, endonentan, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK (D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, atorvastatin, fluvastatin, simvastatin and cerivastatin, (u) fish oils and omega-3-fatty acids, (v) free-radical scavengers/antioxidants such as probucol, vitamins C and E, ebselen, trans-retinoic acid, SOD (orgotein) and SOD mimics, verteporfin, rostaporfin, AGI 1067, and M 40419, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) matrix metalloprotease (MMP) pathway inhibitors such as marimastat, ilomastat, metastat, batimastat, pentosan polysulfate, rebimastat, incyclinide, apratastat, PG 116800, RO1130830 or ABT 518, (y) cell motility inhibitors such as cytochalasin B, (z) antiproliferative/antineoplastic agents including antimetabolites such as purine antagonists/analogs (e.g., 6-mercaptopurine and pro-drugs of 6-mercaptopurine such as azathioprine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), olimus family drugs (e.g., sirolimus, everolimus, tacrolimus, zotarolimus, biolimus, etc.), cerivastatin, flavopiridol and suramin, (aa) matrix deposition/organization pathway inhibitors such as halofuginone or other quinazolinone derivatives, pirfenidone and tranilast, (bb) endothelialization facilitators such as VEGF and RGD peptide, (cc) blood rheology modulators such as pentoxifylline and (dd) glucose cross-link breakers such as alagebrium chloride (ALT-711).

Numerous additional drugs useful for the practice of the present invention are also disclosed in U.S. Pat. No. 5,733,925 to Kunz.

Numerous techniques are available for forming drug-containing layers in accordance with the present invention.

For example, in some embodiments, drug-containing layers are formed using solvent-based techniques. Using these techniques, a drug-containing layer can be formed, for instance, by (a) first providing a solution or dispersion that contains drug(s) and any additional materials (e.g., blending materials, reinforcing elements, rupturing elements, etc.) and (b) subsequently removing the solvent. The solvent that is ultimately selected will contain one or more solvent species, which are generally selected based on their ability to dissolve the drugs that comprise the drug-containing layer, in addition to other factors, including drying rate, surface tension, etc. In certain instances, the solvent is selected based on its ability to dissolve blending materials, if any, as well.

In embodiments where the drug-containing layer is formed from drugs and/or any blending materials having thermoplastic characteristics, a variety of standard thermoplastic processing techniques may be used to form the polymeric region. Using these techniques, a drug-containing layer can be formed, for instance, by (a) first providing a melt that contains therapeutic agent(s) and any supplemental materials (e.g., blending materials, reinforcing elements, rupturing elements, etc.) and (b) subsequently cooling the melt.

In certain embodiments, a solution (where solvent-based processing is employed) or a melt (where thermoplastic processing is employed) is applied to a substrate to form a drug-containing layer. Application techniques include, for example, spin coating techniques, web coating techniques, spraying techniques, dipping techniques, extrusion techniques, techniques involving coating via mechanical suspension including air suspension, ink jet techniques and electrostatic techniques, among others. In certain embodiments, solid reinforcing elements or solid rupturing elements (e.g., solid reinforcing particles or rupturing particles) are applied to the drug-containing layer while the drug-containing layer is in a liquid state, prior to solvent evaporation or melt solidification.

Similar techniques can be used to form rapidly dissolvable layers such as those described above.

With regard to the carbon layer, in some embodiments, a carbon layer is deposited over the drug-containing layer.

For example, U.S. Pat. No. 6,416,820 to Yamada et al. describes a method for forming a carbon hard film that includes vapor depositing a hard film of a carbon material onto a substrate by vacuum deposition of a vaporized, hydrogen-free carbon material, which may be ionized or non-ionized, onto the substrate surface, while irradiating the carbon material with gas cluster ions, generated by ionizing gas clusters to form the film. Yamada et al. report that there is no need to heat the substrate.

T. Kitagawa et al., “Study of Ar Cluster Ion Incident Angle for Super Hard Diamond Like Carbon Film Deposition,” UVSOR Activity report 2003, B1BL8, describe the deposition of super-hard (>50 GPa) DLC thin films with a smooth surfaces and low sp² orbital content at room temperature by Ar gas cluster ion beam (GCIB) assisted deposition using fullerene as the carbon source. See also K Kanda et al. “Characterization of Hard Diamond-Like Carbon Films Formed by Ar Gas Cluster Ion Beam-Assisted Fullerene Deposition,” Jpn. J. Appl. Phys. Vol. 41 (2002) 4295-4298, T. Kitagawa et al., “Optimum Incident Angle of Ar Cluster Ion Beam for Superhard Carbon Film Deposition,” Jpn. J. Appl. Phys. Vol. 43, No. 6B, 2004, pp. 3955-3958 and T. Kitigawa et al., “Near Edge X-Ray Absorption Fine Structure Study for Optimization of Hard Diamond-Like Carbon Film Formation with Ar Cluster Ion Beam,” Jpn. J. Appl. Phys. Vol. 42 (2003) 3971-3975 Part 1, No. 6B, 30 Jun. 2003.

E. Amanatides et al., “Electrical and optical properties of CH₄/H₂ RF plasmas for diamond-like thin film deposition,” Diamond & Related Materials 14 (2005) 292-295, describe the deposition of DLC on PVC foils from CH₄/H₂ using plasma-enhanced chemical vapor deposition (PE-CVD). The authors note that PE-CVD is advantageous because it permits the deposition on polymer substrates, even at room temperature. See also W. S. Choi et al., “Synthesis and characterization of diamond-like carbon protective AR coating,” Journal of the Korean Physical Society, Vol. 45, December 2004, pp. S864-S867 in which DLC films were deposited at room temperature by PE-CVD.

M. Tonosaki et al., in “Nano-indentation testing for plasma-based ion-implanted surface of plastics,” Surf. Coat. Technol., vol. 136, pp. 249-251, 2001, used a filtered cathodic arc as a carbon ion source and supplied bipolar pulses to improve the hardness of amorphous polyolefin. A surface Young's modulus of 25 GPa was reported. In filtered cathodic arc deposition a solid target is evaporated by an arc discharge. A magnetic field is applied to carry ionized particles around a bend, and the ion energy at the substrate can be controlled by applying a bias voltage. Ion bombardment has been shown to improve the quality of films produced by filtered cathodic arc deposition. See M. L. Fulton, “Ion-Assisted Filtered Cathodic Arc Deposition (IFCAD) System for Volume Production of Thin-Film Coatings,” Society of Vacuum Coaters, 42nd Annual Technical Conference Proceedings (1999).

Another example of a deposition-implantation technique is plasma immersion ion implantation-deposition (PIII-D). For instance, J. Y. Chen et al., “Blood compatibility and sp³/sp² contents of diamond-like carbon (DLC) synthesized by plasma immersion ion implantation-deposition,” Surface and Coatings Technology 156 (2002) 289-294 describe the use of plasma immersion ion implantation-deposition (PIII-D) in the fabrication of DLC films on silicon substrates at room temperature. The sp³/sp² ratio (and platelet adhesion) of the film was varied by changing the C₂H₂ to Ar flow ratio during deposition. See also X-M He et al., Journal of Vacuum Science & Technology B: Microelectronics and Nanometer Structures, Volume 17, Issue 2 (March 1999) pp. 822-827, in which DLC films were prepared on low temperature substrates such as poly(methylmethacrylate) (PMMA) using the C₂H₂—Ar plasma immersion ion processing.

In other embodiments, the carbon layer is formed from the materials that comprise the drug-containing layer. For example, a carbon layer may be formed from a drug-containing layer by ion bombardment.

In still other embodiments, the carbon layer is formed from a non-drug-containing layer that is disposed over the drug-containing layer. Materials for forming such non-drug-containing layers may be selected, for example, from suitable organic materials set forth above. For example, a carbon layer may be formed from non-drug-containing layers by ion bombardment. Such embodiments may be advantageous, for example, where the drug is very valuable and where it is desirable to minimize drug destruction upon carbon layer formation.

An example of an ion bombardment technique is plasma immersion ion implantation (PIII). In such techniques, ions generated in a plasma are bombarded onto an organic layer (e.g., a drug-containing layer or a non-drug-containing layer that is disposed over a drug-containing layer).

Where insulators are being bombarded, problems can be encountered as a result of a potential drop across the sample, which may be so severe that no implantation occurs. This problem has been explained in terms of capacitance and surface charging effects, which lead, for example, to electrical arcing and decreased ion energy. To address this problem, so-called “mesh assisted” techniques have been employed in which a conductive grid is placed over the sample and in electrical contact with an underlying conductive substrate holder. Consequently, ions are accelerated toward the grid and pass through the holes where they are implanted into the insulator surface. The size of the grid holes is adjusted to optimize ion energy and dose uniformity. See e.g., P. K. Chu, “Recent developments and applications of plasma immersion ion implantation,” J. Vac. Sci. Technol. B 22(1), January/February 2004, 289-296. Such grids are known to create shadow effects, which can be addressed by moving the sample relative to the grid (e.g., either during implantation or between implantation steps). On the other hand, in some embodiments, shadow effects may be used to create rupture lines in the carbon layer, which are forced to follow the shadow effect pattern, whereas normal rupture lines would be random in case of a more homogeneous treatment. Further information on mesh-assisted PIII can be found, for example, in P. K. Chu, “Recent developments and applications of plasma immersion ion implantation,” J. Vac. Sci. Technol. B 22(1), January/February 2004, 289-296, R. K. Y. Fu et al., “Effects of mesh-assisted carbon plasma immersion ion implantation on the surface properties of insulating silicon carbide ceramics,” J. Vac. Sci. Technol. A 22(2), March/April 2004, 356-360; R. K. Y. Fu et al., “Influence of thickness and dielectric properties on implantation efficacy in plasma immersion ion implantation of insulators,” J. Appl. Phys., Vol. 95, No. 7, 1 Apr. 2004, 3319-3323.

Bombarding species for PIII include, for example, inert species such as argon, helium and nitrogen ions, among others. Typical working pressures range from 1×10⁻⁴ Pa to 1×10⁻³ Pa. Plasma is generated by a radio frequency generator operating at 13.56 MHz. Applied voltages during PIII of biodegradable polymeric regions may range, for example, from 10 kV to 100 kV, with pulse duration ranging from 1-100 μs at a frequency ranging from 10 to 1000 Hz. In general, the ratio of sp³ hybridized carbon to sp² hybridized carbon increases with increasing dose. Typical dosages may range, for example, from 10¹⁵ to 10¹⁷ ions per cm², among other possibilities. An increase in energy will generally result in an increase in thickness of the carbon layer that is formed. Typical energies may range, for example, from 10 keV to 50 keV, among other possibilities. Additional information regarding the treatment of organic surfaces, specifically polymeric surfaces, using PIII to form carbon layers can be found in Pub. No. US 2007/0191923 to Weber et al.

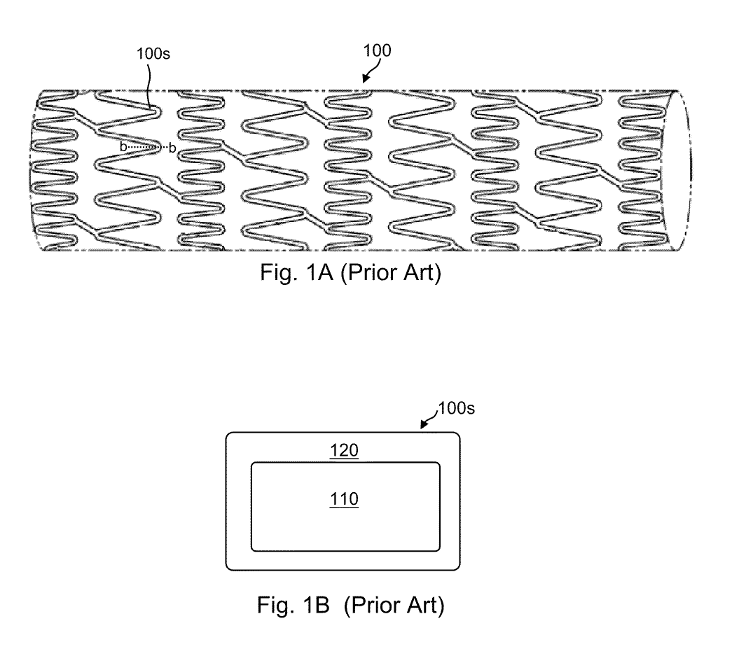

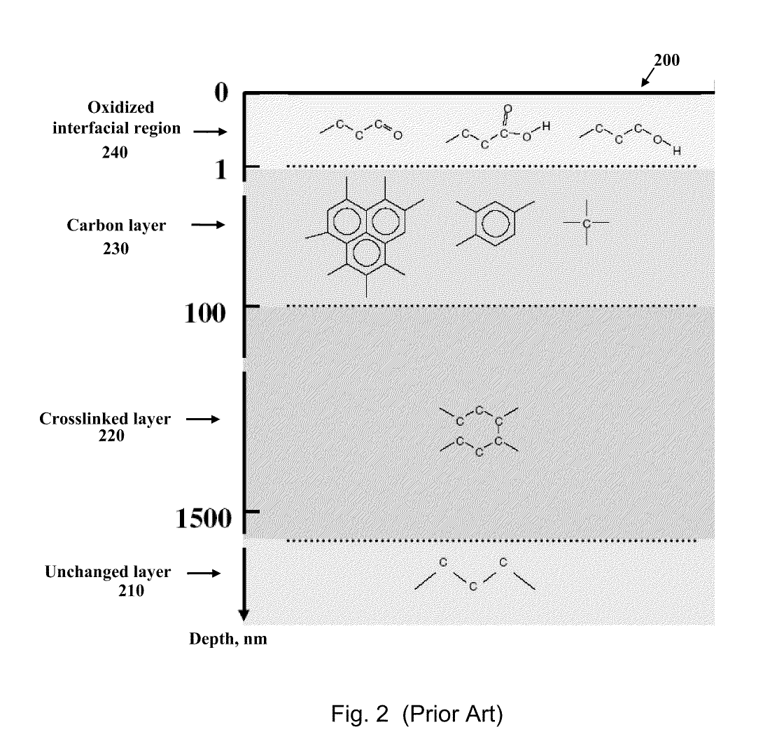

Turing now to FIG. 10A, a stent body 100, analogous in design to that shown in FIG. 1A, is shown which comprises various struts 100s. Unlike the stent of FIG. 1A, however, stent body 100 is constructed to in accordance with the present invention. For example, FIG. 10B is a schematic cross-sectional view of a stent strut 100s taken along line a-a of FIG. 10A. As seen from FIG. 10B the stent strut 100s comprises a substrate 110, a drug-containing layer 120 and a carbon layer 130.

Where line-of-sight deposition and/or implantation techniques are employed to create the carbon layer 120, the carbon layer 120 can be formed on both the inner and outer surfaces of the stent substrate 110, for instance, by moving (e.g., rotating, tilting, etc.) the stent substrate 110 in a continuous or stepwise fashion during processing. The carbon layer 120 may be formed on the inner surface of the stent substrate 110, because species for deposition/ion implantation are allowed to pass from the exterior to the interior of the device through the open spaces 100w that are present between the struts 100s.

In the event that it is desired to form a carbon layer only on the outer surface of the stent, the stent may be mounted on a mandrel or another support which acts to prevent species from passing through the open spaces 100w and striking the interior surface of the device. A carbon layer may be formed only on the inner surface of the stent by masking the inner surface of the stent after forming a carbon layer over the entire device, followed by etching and mask removal.

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable medical device comprising (a) a substrate, (b) a drug-containing layer over the substrate, said drug-containing layer comprising a drug and a rupturing element, and (c) a carbon layer disposed over said drug-containing layer, wherein said rupturing element acts to rupture the carbon layer during or after implantation or insertion of said device.

2. The implantable or insertable medical device of claim 1, wherein said rupturing element ruptures the carbon layer upon application of a compressive force to the drug-containing layer and the carbon layer during implantation or insertion of said device.

3. The implantable or insertable medical device of claim 2, wherein said rupturing element is a hard, sharp structural element which ruptures the carbon layer upon application of a compressive force to the drug-containing layer and the carbon layer.

4. The implantable or insertable medical device of claim 3, comprising a plurality of said hard, sharp structural elements.

5. The implantable or insertable medical device of claim 4, wherein said plurality of said structural elements comprise sharp metallic or non-metallic inorganic particles.

6. The implantable or insertable medical device of claim 1, wherein said rupturing element expands and ruptures the carbon layer upon exposure to bodily fluid.

7. The implantable or insertable medical device of claim 6, wherein said rupturing element is selected from a metallic element that expands upon corrosion and a hydrogel element that expands upon aqueous fluid uptake.

8. The implantable or insertable medical device of claim 1, wherein the substrate is a metallic substrate.

9. The implantable or insertable medical device of claim 1, wherein the device is a stent.

10. The implantable or insertable medical device of claim 1, wherein the carbon layer is a diamond-like carbon layer.

11. The implantable or insertable medical device of claim 10, wherein said diamond-like carbon layer comprises an sp³ fraction of 50% or more.

12. The implantable or insertable medical device of claim 10, wherein said diamond-like carbon layer is either vapor deposited or formed from carbon atoms that were previously part of said drug-containing layer.

13. A medical device comprising (a) a substrate, (b) a drug-containing layer over the substrate, said drug-containing layer comprising a drug and a reinforcing element, and (c) a carbon layer disposed over said drug-containing layer, wherein said reinforcing element protects said carbon layer by preventing compression of the drug-containing layer upon application of a compressive force to the drug-containing layer.

14. The implantable or insertable medical device of claim 13, wherein said reinforcing element is selected from a fiber mesh, a screen, and a porous membrane.

15. The implantable or insertable medical device of claim 13, comprising plurality of reinforcing elements.

16. The implantable or insertable medical device of claim 15, wherein the reinforcing elements are circular or oval in cross-section.

17. The implantable or insertable medical device of claim 15, wherein the reinforcing elements are spheroidal reinforcing elements.

18. The implantable or insertable medical device of claim 13, further comprising a rapidly dissolvable layer disposed over the carbon layer.

19. An implantable or insertable medical device comprising (a) a substrate, (b) a drug-containing layer over the substrate, said drug-containing layer comprising a drug, (c) a carbon layer disposed over said drug-containing layer, and (d) a rapidly dissolvable organic layer disposed over the carbon layer.

20. The implantable or insertable medical device of claim 19, wherein the rapidly dissolvable organic layer comprises a material selected from a polysaccharide and a protein, a glycoprotein and a fatty acid alkyl ester.

21. The implantable or insertable medical device of claim 19, wherein the rapidly dissolvable organic layer comprises a plurality of sharp hard particles.
